(19) United States
(12) Patent Application Publication
(10) Pub. No.: US 2016/0029965 A1
(43) Pub. Date: Feb. 4, 2016

(54) ARTIFACT AS A FEATURE IN NEURO DIAGNOSTICS

(71) Applicants: Adam J. SIMON, Yardley, PA (US); Cerora, Inc., Bethlehem, PA (US)

(72) Inventor: Adam J. SIMON, Yardley, PA (US)

(21) Appl. No.: 14/777,012

(22) PCT Filed: Mar. 12, 2014
(86) PCT No.: PCT/US14/23960

§ 371 Date: Sep. 15, 2015

Related U.S. Application Data
(60) Provisional application No. 61/792,274, filed on Mar. 15, 2013.

(51) Int. Cl. A61B 5/00; A61B 5/16; A61B 5/0476

(52) U.S. Cl. CPC A61B 5/7203; A61B 5/7275; A61B 5/7289; A61B 5/0476; A61B 5/16; A61B 5/6897; A61B 5/4088; A61B 2562/0204

(57) ABSTRACT

A multi-modal physiological assessment device and method enables the simultaneous recording and then subsequent analysis of multiple data streams of biological signal measurements to assess the health and function of the brain. Means and methods are provided to identify and leverage artifact samples within ID and 2D bio signal data streams to help create more accurate predictors and classifiers of brain health states and conditions. One sensor's data is used to gate the relevant portion of another bio sensor's data in order to reduce the noise and increase the signal-to-noise ratio. This is a form of phase locking for multimodal data streams for brain health assessment.

### CROSS REFERENCE TO RELATED APPLICATIONS

This application claims benefit of Provisional Application No. 61/792,274 filed Mar. 15, 2013. The content of that patent application is hereby incorporated by reference in its entirety.

### TECHNICAL FIELD

The invention relates to diagnosis and analysis of brain health through the use of activated tasks and stimuli in a system to dynamically assess one's brain state and function.

### BACKGROUND

Normal functioning of the brain and central nervous system is critical to a healthy, enjoyable and productive life. Disorders of the brain and central nervous system are among the most dreaded of diseases. Many neurological disorders such as stroke, Alzheimer's disease, and Parkinson's disease are insidious and progressive, becoming more common with increasing age. Others such as schizophrenia, depression, multiple sclerosis and epilepsy arise at younger age and can persist and progress throughout an individual's lifetime. Sudden catastrophic damage to the nervous system, such as brain trauma, infections and intoxications can also affect any individual of any age at any time.

Most nervous system dysfunction arises from complex interactions between an individual's genotype, environment and personal habits and thus often presents in highly personalized ways. However, despite the emerging importance of preventative health care, convenient means for objectively assessing the health of one's own nervous system have not been widely available. Therefore, new ways to monitor the health status of the brain and nervous system are needed for normal health surveillance, early diagnosis of dysfunction, tracking of disease progression and the discovery and optimization of treatments and new therapies.

Unlike cardiovascular and metabolic disorders, where personalized health monitoring biomarkers such as blood pressure, cholesterol, and blood glucose have long become household terms, no such convenient biomarkers of brain and nervous system health exist. Quantitative neurophysiological assessment approaches such as positron emission tomography (PET), functional magnetic resonance imaging (fMRI) and neuropsychiatric or cognition testing involve significant operator expertise, inpatient or clinic-based testing and significant time and expense. One potential technique that may be adapted to serve a broader role as a facile biomarker of nervous system function is a multi-modal assessment of the brain from a number of different forms of data, including electroencephalography (EEG), which measures the brain's ability to generate and transmit electrical signals. However, formal lab-based EEG approaches typically require significant operator training, cumbersome equipment, and are used primarily to test for epilepsy.

Alternate and innovative biomarker approaches are needed to provide quantitative measurements of personal brain health that could greatly improve the prevention, diagnosis and treatment of neurological and psychiatric disorders. Unique multi-modal devices and tests that lead to biomarkers of Parkinson's disease, Alzheimer's disease, concussion, Autism Spectrum Disorder, and other neurological and neuropsychiatric conditions is a pressing need.

### SUMMARY

The invention relates to an analysis method for pre-processing biological sensor data before conducting spectral, non-linear, wavelet, time series or other signal processing on the biological sensor data from one of a plurality of different and independent biological sensor data streams. In this pre-processing step, areas of artifact are identified and flagged. In the present invention, rather than ignore these areas of artifact, features of the artifact are analytically characterized as candidate predictor variables for predictive statistical models for analysis of the biological sensor data streams.

An embodiment of the invention includes using the areas of artifact or areas of interest in one biological sensor data stream as a time marker for analysis of another biological sensor data stream. A flagged artifact or area of interest in one biological sensor data stream may be used to temporally (in time) gate data from another biological sensor data stream to enhance the signal to noise ratio within the gated biological sensor data stream.

In exemplary embodiments, the artifact may be flagged manually or automatically. In the case of manual flagging, mouse clicks on one or more buttons or key strokes on one or more keys of a keyboard may be used to mark times at the beginning and end of a time frame or period of interest. In this way, the “start” click and “stop” click on the screen buttons provide reference markers in time for both the beginning and end of the interesting data from the brainwave sensor, eye tracker, pulse oximeter, blood perfusion microphone, or balance accelerometers, in each case another assessment modality besides the mouse clicks or keyboard stoke when they are conducting the task. Alternatively, in the case of automatic flagging, the system could analyze an acoustic microphone time series to automatically determine when the first value is read symbolizing the “start” fiduciary time point and when a last value is read symbolizing the “stop” fiduciary time point during oral testing of a patient without the need to press a mouse or other fiduciary time markers. In this example, analysis of the audio microphone data stream could determine and mark the beginning and end of the EEG brainwave sensor, eye tracker, pulse oximeter, blood perfusion microphone, and balance accelerometer data to be analyzed, acting as a sort of automatic inclusion “gate” which decreases the noise and increases the signal to noise ratio.

The invention also includes additional steps to specifically analyze the artifact data for putative predictor variables to create additional features to be used as putative diagnostic information alone or to be used in development of multi-variate predictive statistical models.

Several approaches may be used to identify and flag areas of artifact in the biological sensor data. For example, the number N of artifacts in a block of data may be extracted or the rate of artifacts noted as the number N of artifacts per unit of time, such as per second, minute or hour. In another embodiment, the set of locations of artifacts within the one-dimensional data stream {xᵢ} or two-dimensional data stream {(xᵢ, yᵢ)} is determined and recorded to a storage device. In another embodiment, the central value (xₗ−x_{f})ᵢ/2 of an artifact is determined for a one-dimensional data stream or the equivalent for each dimension in a two-dimensional data stream. In yet another embodiment, the weighted value of the amplitude within the window of the artifact is used to understand how large or small the artifact is in relation to other sorts of artifact.

Identifying and flagging areas of artifact in accordance with the invention may also include distribution of extracted lengths L of the artifact in terms of individual samples from a time series, which can be calculated for each artifact by Lᵢ=xₗ−x_{f}. Determination of the mean value of the signal over the artifact region may also be used. Another embodiment includes the nonlinearly calculated median value of the signal within an artifact window taken as the central value after an ascending or descending sort of the values in the sensor data stream has occurred.

Another embodiment includes calculating the standard deviation and higher order moments (3^{rd} order skewness and fourth order kurtosis) of the distribution of sample amplitudes from the artifact zones or epochs. After each of these is calculated on a per artifact basis, the number of artifacts N, the distribution of artifacts, and the various moments of the distribution of various artifacts can thus be calculated and each of these can be extracted as another candidate diagnostic predictor variable.

Of particular interest in accordance with the present invention is the evaluation of the relative position of artifacts in a data stream of interest relative to the presentation of external sensory and cognitive stimuli, or when a physical motion challenge is presented. Alternatively, the relative position of an artifact in one data stream when compared to other features of interest (good/correct answers or perhaps bad) in other data streams that have been temporally synchronized may be considered.

### BRIEF DESCRIPTION OF THE DRAWINGS

Embodiments of the invention can be better understood with reference to the following drawings, of which:

FIG. 1 is an artificially created panel of illustrative biosignal data streams to show the diversity of a multi-modal assessment.

FIG. 2A is a graphical representation of an original biosignal data stream from a biosignal transducer before artifact detection and signal pre-processing.

FIG. 2B is a graphical representation of the biosignal data stream from the biosignal transducer shown in FIG. 2A after artifact detection and signal pre-processing.

FIG. 3A is a graphical representation of a raw biosignal data stream from a biosignal transducer before artifact detection and signal pre-processing.

FIG. 3B is a graphical representation of the same biosignal data stream from the biosignal transducer shown in FIG. 3A after artifact detection and signal pre-processing.

FIG. 4A is a graphical representation of a raw biosignal data stream from a biosignal transducer in the form of a microphone recording of the voice of a subject before artifact detection and signal pre-processing when the subject said “one”, then paused, then said “two.”

FIG. 4B is a graphical representation of a raw biosignal data stream from a biosignal transducer in the form of a microphone recording of the voice of a subject before artifact detection and signal pre-processing when the subject said “one”, then paused and said “uummm”, then said “two.”

### DETAILED DESCRIPTION OF ILLUSTRATIVE EMBODIMENTS

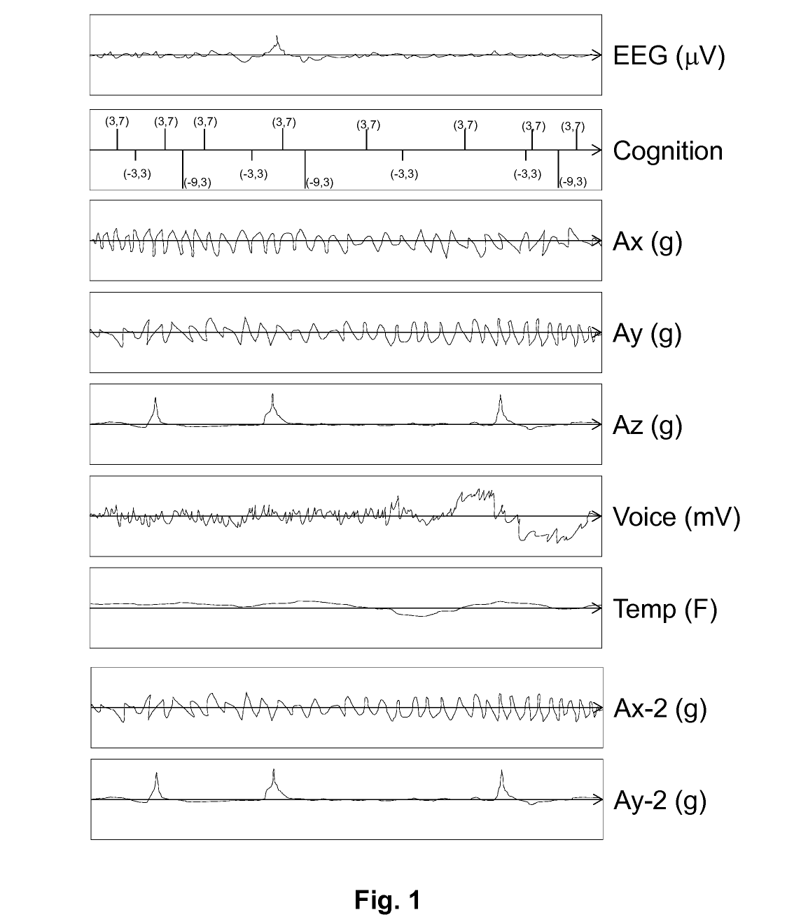

The invention will be described in detail below with reference to FIGS. 1-4. Those skilled in the art will appreciate that the description given herein with respect to those figures is for exemplary purposes only and is not intended in any way to limit the scope of the invention. All questions regarding the scope of the invention may be resolved by referring to the appended claims.

### DEFINITIONS

By “electrode to the scalp” we mean to include, without limitation, those electrodes requiring gel, dry electrode sensors, contactless sensors and any other means of measuring the electrical potential or apparent electrical induced potential by electromagnetic means.

By “monitor the brain and nervous system” we mean to include, without limitation, surveillance of normal health and aging, the early detection and monitoring of brain dysfunction, monitoring of brain injury and recovery, monitoring disease onset, progression and response to therapy, for the discovery and optimization of treatment and drug therapies, including without limitation, monitoring investigational compounds and registered pharmaceutical agents, as well as the monitoring of illegal substances and their presence or influence on an individual while driving, playing sports, or engaged in other regulated behaviors.

A “medical therapy” as used herein is intended to encompass any form of therapy with potential medical effect, including, without limitation, any pharmaceutical agent or treatment, compounds, nutraceuticals, biologics, medical device therapy, exercise, biofeedback or combinations thereof.

By “EEG data” we mean to include without limitation the raw time series, any spectral properties determined after Fourier transformation, any nonlinear properties after non-linear analysis, any wavelet properties, any summary biometric variables and any combinations thereof.

A “sensory and cognitive challenge” as used herein is intended to encompass any form of sensory stimuli (to the five senses), cognitive challenges (to the mind), and other challenges (such as a respiratory CO₂ challenge, virtual reality balance challenge, hammer to knee reflex challenge, etc.).

A “sensory and cognitive challenge state” as used herein is intended to encompass any state of the brain and nervous system during the exposure to the sensory and cognitive challenge.

An “electronic system” as used herein is intended to encompass, without limitation, hardware, software, firmware, analog circuits, DC-coupled or AC-coupled circuits, digital circuits, FPGA, ASICS, visual displays, audio transducers, temperature transducers, olfactory and odor generators, or any combination of the above.

By “spectral bands” we mean without limitation the generally accepted definitions in the standard literature conventions such that the bands of the PSD are often separated into the Delta band (f<4 Hz), the Theta band (4<f<7 Hz), the Alpha band (8<f<12 Hz), the Beta band (12<f<30 Hz), and the Gamma band (30<f<100 Hz). The exact boundaries of these bands are subject to some interpretation and are not considered hard and fast to all practitioners in the field.

By “calibrating” we mean the process of putting known inputs into the system and adjusting internal gain, offset or other adjustable parameters in order to bring the system to a quantitative state of reproducibility.

By “conducting quality control” we mean conducting assessments of the system with known input signals and verifying that the output of the system is as expected. Moreover, verifying the output to known input reference signals constitutes a form of quality control which assures that the system was in good working order either before or just after a block of data was collected on a human subject.

By “biomarker” we mean an objective measure of a biological or physiological function or process.

By “biomarker features or metrics” we mean a variable, biomarker, metric or feature which characterizes some aspect of the raw underlying time series data. These terms are equivalent for a biomarker as an objective measure and can be used interchangeably.

By “non-invasively” we mean lacking the need to penetrate the skin or tissue of a human subject.

By “diagnosis” we mean any one of the multiple intended use of a diagnostic including to classify subjects in categorical groups, to aid in the diagnosis when used with other additional information, to screen at a high level where no a priori reason exists, to be used as a prognostic marker, to be used as a disease or injury progression marker, to be used as a treatment response marker or even as a treatment monitoring endpoint.

By “electronics module” or “EM” or “reusable electronic module” or “REM” or “multi-functional biosensor” or “MFB” we mean an electronics module or device that can be used to record biological signals from the same subject or multiple subjects at different times. By the same terms, we also mean a disposable electronics module that can be used once and thrown away which may be part of the future as miniaturization becomes more common place and costs of production are reduced. The electronics module can have only one sensing function or a multitude (more than one), where the latter (more than one) is more common. All of these terms are equivalent and do not limit the scope of the invention.

By “bio signals” or “biosignals” we mean any direct or indirect biological signal measurement data stream which either directly derives from the human subject under assessment or indirectly derives from the human subject. Non-limiting examples for illustration purposes include EEG brainwave data recorded either directly from the scalp or contactless from vicinity of the scalp, core temperature, physical motion or balance derived from body worn accelerometers, gyrometers, and magnetic compasses, the acoustic sound from a microphone to capture the voice of the individual, the stream of camera images from a front facing camera, the heart rate, heart rate variability and arterial oxygen from a pulse oximeter, the dermal skin conductance measured along the skin, the cognitive task information recorded as keyboard strokes, mouse clicks or touch screen events. There are many other bio signals to be recorded as well.

### Signal Pre-Processing as Part of a Larger Analytical Work Flow

The system and signatures of the present invention include approaches to analyze raw signal data streams for artifacts and noise and to turn them into potentially useful diagnostic information on which brain health assessment classifiers and signatures can be created. In particular, the present invention describes how to analyze raw signal data to first identify characteristics that appear to be or are similar to artifacts and secondly how to quantify those artifacts in such a way that they become additional extracted features or useful diagnostic information. Finally, predictive models built on artifacts become a part of the present invention as well.

Consider a human subject being scanned for their brain health with a multi-modal diagnostic system. The equipment collects numerous parallels streams of bio signal data from the human subject. Multiple transducers both stimulate and record the physiological response of the brain and the body in order to assess its health and function. Central to the system is the ability to directly record brainwave activity from an electrode placed non-invasively on or near the scalp. Moreover, additional information on brain health and function can be derived from transducers that measure position and motion (such as from a multi-axis (e.g. 9 axis) combination accelerometer, gyrometer and/or magnetic compass), temperature, cardiovascular properties like heart rate, heart rate variability, and arterial oxygen, as well as cognitive information, speech quality and processing, eye movement and saccade, cerebral blood perfusion measured by a small micro phone placed in the ear canal, and dermal surface skin conductance (i.e. galvanic skin conductance) to name a few non-limiting additional biological signal measurement data stream examples. It is often necessary and desirable to bring the system to the human subject getting out of the hospital or doctor's office and enabling data collection in the home or at the sports field or in the combat theater, thus providing accessibility to the brain health and function assessment from a lightweight and portable form factor.

A common challenge during the acquisition and analysis of bio signal measurement data streams is the evaluation of the digital data streams to identify those areas that are perceived to be contaminated with various artifacts and identify those areas of data that are perceived as having good information content. In one particular embodiment of the invention, one of the first tasks conducted on a bio signal data stream is signal pre-processing to clearly delineate areas of perceived artifact from areas of perceived information. This determination is typically done at the sample level in a 1D digital data stream and the pixel level for 2D digital data streams or voxel level for 3D digital data streams. Perceived artifacts can occur for many different reasons; some are measurement related, while some are intrinsic to the biological variability between human subjects or within the same human subject but dependent on uncontrolled variables like the time of day or the hydration state of the individual. This can also be true for heart related bio signals, weight related bio signals (where morning weight and evening weight are systematically different), actigraphy levels (gathered from accelerometer based measurements) as well as brain related bio signals.

In the case of brain related bio signals, each bio signal data stream can inadvertently be mixed or combined with noise of various and different sources. Noise typically falls into either of two classes: (i) systematic or measurement noise, commonly due to the equipment and detection methodology; or (ii) biological noise due most frequently to the individual variability and characteristics of each human or animal. For the former, there are methods of understanding what certain artifacts look like. Typically measurement artifacts include, but are not limited to, motion (in a camera system for instance), heart beat (when trying to measure brainwaves), insufficient mechanical joint integrity (when trying to measure heart or brain electrophysiology), ambient acoustic noise (when trying to measure and record a human subjects speech on the side line of a football game with a crowd in the stands and a big play taking place on the field leading to an enormous crowd based cheer) to mention but a few non-limiting examples.

If one examines closely the output from the various bio sensors and transducers place on or near a human subject, one can see the quantitative output from each sensor or transducer, after analog to digital conversion by an ADC into a discrete flow of digital information. FIG. 1 schematically illustrates the real-time synchronously collected output from nine sensors and transducers (artificial data created for illustration purposes only), each a different bio signal stream. From the top of FIG. 1, one sees the electroencephalogram or EEG in micro-volts (μV) plotted on the y-axis as a function of time t along the x-axis. Typical sample times range from 100 samples per second to 10,000 samples per second. In the second trace down, neuropsychological “Cognition” data is illustrated in a plot where discrete response “events” to computer neuropsychological testing are being captured either as (i) key strokes on a keyboard or as (ii) mouse clicks of the cursor along the surface of the video display with a position (x,y) on the video monitor's screen at a given time t or alternatively (iii) on a touch screen display as touch “events” where the touch location (x,y) is much like a mouse click location and is recorded as (x,y) spatial points at a given instant in time t to form an (x, y, t) two-dimensional time series. In the next three traces down FIG. 1, (third, fourth and fifth from the top) one sees three independent traces from a 3-axis digital accelerometer or a 3-axis analog accelerometer after passing through an ADC. Acceleration is often expressed as a fraction or multiple of the gravitational acceleration constant g=9.8 meters/second. In the sixth trace from the top (or fourth from the bottom), one can see an acoustic microphone recording trace, typically sampled at either 8 or 16 bits per sample and from 5 ksam/sec to 8 ksam/sec to 12 or even as high as 16 ksam/sec, 20 ksam/sec or 44.2 ksam/sec. In the third trace from the bottom, the temperature T in Fahrenheit of the human subject is plotted across time to investigate if any of the sensory stimulations or cognitive tasks is having an effect on core body temperature or vice versa if an infection is elevating body temperature and this is in fact affecting cognition. Lastly, the bottom two traces exemplify either a two axis accelerometer or two of three axes of accelerometer data from a second REM, perhaps located on the trunk at the chest or small of the back, or on a limb around the wrist or perhaps ankle. If well synchronized and registered in time, the multiple streams of bio signals enable several clever and interesting techniques of data acquisition and analysis.

Gating a First Sensor's Data Stream from a Second Sensor's Data Stream

A first sensor's information can be used to gate periods of interest in a second sensor's data stream. As a non-limiting example, consider a situation where a human subject is reading numbers from (i) the King-Devick (K-D) Ophthalmological test cards (Oride et al 1986, Amer J Opto Physiol Optics, Reliability study of the Pierce and King-Devick Saccade Tests), (ii) the Developmental Eye Movement (DEM) test cards, or (iii) a Cerora proprietary improvement on the DEM cards as non-limiting examples. The conventional approach according to the published literature is for a test administrator to time the participant with a stop watch manually (starting with the first number read on the card and stopping with the last number read on each card) and add the total time for the three test cards with minimal errors on a sheet of paper (for the K-D test). An improvement upon this is an embodiment of the invention whereby the test subject is instructed to click a mouse on a start button on the screen to initiate the beginning of a new card and to click a stop button just after finishing the last number on the card. In this way, the start click and stop click on the screen buttons provide reference markers in time for both the beginning and end of the interesting data from the brainwave sensor or accelerometers, in each case another assessment modality than the mouse clicks or keyboard stoke when they are conducting the task. Alternatively, one could analyze the acoustic microphone time series and automate determining when the first number is read on a given card and when the last number is read, without the need to press a mouse or other fiduciary time markers. In this embodiment, analysis of the audio microphone data stream could itself mark the beginning and end of the EEG, pulse oximeter, gaze tracker, galvanic skin conductance, and accelerometer data to be analyzed, acting as a sort of inclusion “gate.” This is similar in spirit to how a Flow Activated Cell Sorter (FACS) can use the FCS or SSC channels or the FCS by SSC plane to gate on certain cell types and then only look for various fluorescence signals in the FL1 and FL2 channel for those that meet a gate requirement in independent measurement channels (in this example the FCS and SSC channels). The invention now utilizes multiple channels or independent modalities of information to achieve more selective and gated signal analysis.

Unlike FACS scanners or any other technology that the inventors are aware of, an embodiment of the present invention includes the inclusive or exclusive gating on one bio-sensor modality of information in time (bio signal data stream) based on a 2^{nd} independent modality of information (2^{nd} bio signal data stream) for brain health assessment, diagnosis, evaluation, and management. In the above non-limiting illustrative case, the microphone modality is used to identify the beginning and end of each King-Devick test card so that only the EEG data or perhaps the accelerometer data (a different modality) is evaluated when the subject is conducting the actual task. This method of gating reduces noise (evaluation of time series and samples when something pertinent is not taking place) and thus increases the signal to noise ratio.

In an alternative embodiment, one could study the integrated 3-axis accelerometer data set to trace a point in 3D space as a function of time (x, y, z, t) and examine the surface on which a subject traces their Center of Mass in a given time. This surface could then have its center of mass, center of gravity or centroid determined by standard analytical techniques. The invention uses one modality (key strokes or more preferably acoustic microphone information) to gate an independent 2^{nd} modality bio signal data stream (EEG data, pulse oximetry, cerebral blood perfusion with an ear canal mounted microphone, or accelerometer data) from synchronously collected bio signal streams for brain health assessment. Although it is not essential to the present invention to have EEG as one modality, it is preferable to have at least one channel of EEG present directly recording brainwave activity.

For neuropsychiatric conditions, an exemplary embodiment of the present invention uses Galvanic skin conductance or Dermal Skin Resistance as a means of objectively assessing mood whereby the sweat secreted from skin glands shifts the electrical conductance when one becomes agitated, nervous or upset. Cool, calm, and collected is usually represented by dry skin with low conductance and high impedance.

### Artifact Detection in Raw 1-Dimensional (1D) or 2-Dimensional (2D) Signal Streams

The invention starts by implementing standard methods of artifact detection. For instance, certain artifacts can have a characteristic shape or pattern which can be captured in a kernel and then convolved with the 1D or 2D system to look for locations in the 1 or 2 dimensions where the Kernel matches the bio signal. Alternatively, if a signal is known to always be changing in a human subject, such as their heart ECG or brain EEG, then any instances in a time series that are repetitively consistent beyond statistical test, such as a section of signal that has the same value for 10 or 15 samples when anything after 5 is considered suspicious. In this case, one could run a rolling average conditional test and anywhere the value does not change within the window width, then that section from beginning to end could be flagged as an artifact, often called a “drop-out” artifact as if the true signal were dropped out of the recorded bio-signal creating the artifact. This could equally work for saturated signals whereby the signal appears pinned high to an upper rail or low to a lower rail of an amplifier and does not vary over too many samples. This is often called a so-called “rail” artifact.

A third type of artifact could be due to uncontrolled biological processes. For instance, imagine a human subject getting a heart ECG assessment when they have a cold and they randomly or inadvertently cough or sneeze during measurement and recording by the bio-sensor. This would lead to a violent body shake during the cough or sneeze which could lead to motion of the electrodes hooked to the skin to record the heart electrophysiology, which would then lead to varying electrical impedance and thus a poor recording during that time which would show a lot of variation but that variation is not due to the heart's electrical signals but rather due to the cough or sneeze which induced electrode motion and variable impedance. The impedance of the connection could physically change, or the electrical signal propagation could be disrupted. All these examples of noise or artifact need to be identified a priori, typically visually by a subject matter expert first, and then implemented into a pattern recognition, semi-automated, or automated artifact detection pre-processing algorithm.

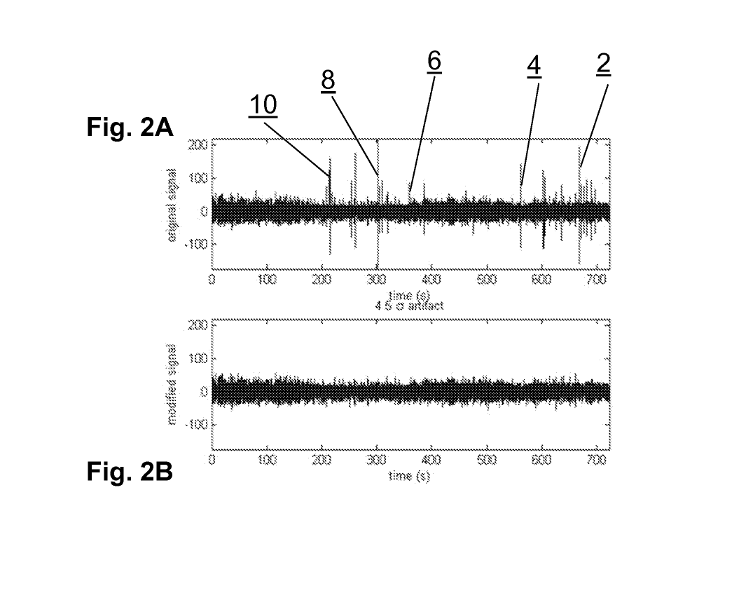

In FIG. 2A, one can see a graphical representation of a single lead brainwave EEG data stream from position Fp1 just above the left eye on the forehead. One can easily observe several larger fluctuations in signal amplitude that appear to be non-biological slew rates as defined as the change in voltage as a function of time or dV/dt. After long and careful observation of hundreds to thousands of hours of human bio signal data, one can estimate well the typical slew rate for a human brain as measured on the surface of the skull. Thus, at 2, 4, 6, 8, and 10 in FIG. 2A, large non-biological slew rates can be seen. In FIG. 2B, an artifact detection pre-processing algorithm identified any fluctuations larger than 4.5 standard deviations from the mean value (in this case centered on zero) of the trace in FIG. 2A and automatically removed them as can be seen in lower FIG. 2B.

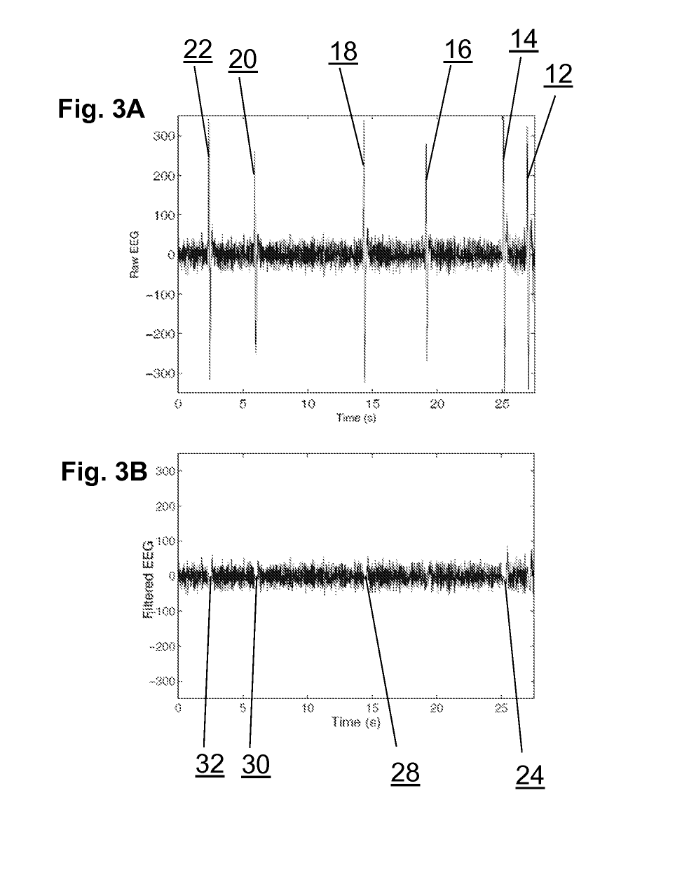

In a similar spirit, FIG. 3A presents a graphical representation of the raw single lead brainwave EEG data stream from position Fp1 just above the left eye on the forehead of a different subject. One can easily observe six large fluctuations in signal amplitude that appear to be non-biological slew rates as defined above. Thus, the artifacts in the form of slew blips 12, 14, 16, 18, 20, and 22 in FIG. 3A can be removed as shown in FIG. 3B, after an artifact detection pre-processing algorithm identified any fluctuations larger than a cut-point threshold, in this particular embodiment, 4.5 times the standard deviations from the mean value (in this case centered on zero) of the trace in FIG. 3A, and removed them as can be seen in lower FIG. 3B. Gap 24 corresponds to where blip 14 was located; gap 28 is where peak 18 was located; and gaps 30 and 32 are where peaks 20 and 22 used to be located, respectively.

### Artifact Sample Data as a Complementary Data Source for Feature Extraction

An embodiment of the present invention reveals itself at this point in the workflow. Rather than just identify or flag with a binary marker the bad or artifactual samples in the signal data stream and skip over those “bad” samples when analyzing the data for content, the present invention takes a completely alternate point of view. In the present invention, additional steps in the analysis are specifically undertaken to focus on analyzing the artifactually flagged samples as a complementary set of data points which can be analyzed to create additional extracted features to be used as putative diagnostic information alone, or in the development of multi-variate predictive statistical models with features extracted from the conventionally non-artifactual regions.

In one particular embodiment, the number N of artifacts in a block of data is extracted. This could be relevant if a tic or repetitive task is noted in the objective data streams in one condition and not in other classifications. An illustrative example of this would be if someone blinked their eyes at a much higher frequency or rate in one condition relative (say Alzheimer's disease) relative to another condition B (say Mild Cognitive Impairment). In addition, the count N or percentage of artifact samples P in a given block can become a candidate predictor variable. This can be seen in Table 1 where four blocks of Eyes Open (EO) or Eyes Closed (EC) data are present. It is apparent that when the eyes are open EO (the second and fourth row in the table) the number N of artifacts is large (20 or 22) whereas when Eyes Closed (EC) there are only 1 or 5 artifacts, typically eye blinks.

TABLE 1
Artifact analysis of ajs_22jun2009 EC/EO data blocks.

| Filename | Number N Artifacts | % of artifact samples |
|---|---|---|
| 90622080729 | 1 | 0.004439 |
| 90622081101 | 20 | 0.088137 |
| 90622081431 | 5 | 0.022044 |
| 90622081801 | 22 | 0.097036 |

In another embodiment, the set of locations of the artifacts within the 1D {xᵢ} or 2D data stream {(xᵢ, yᵢ)} is determined and recorded to a mass storage device. This set can be annotated in one embodiment by the first artifactual sample x_{f} and the last artifactual sample xₗ where the set is the pairwise combination of sample locations along the data stream axis x, in this case, denoted {(x_{f}, xₗ)ᵢ} for a 1D data stream in which x is the independent variable (which could be time t if a time series or the first of two Cartesian coordinates in a planar geometrical space) and {(x_{f},y_{f}|xₗ,yₗ)ᵢ} for a 2D data stream like a series of video rate images from the front facing camera of a personal computer, tablet computer or smartphone, at either 30 Hz if counting by frame or 60 Hz if counting by field. Another embodiment is the determination of the central value (xₗ−x_{f})ᵢ/2 of an artifact for a 1D data stream or the equivalent for each dimension independently in a 2D data stream. Again, the most common 2D data stream is a movie from an video rate image sensor (as in a stream of images were each image has a pixel at (x,y) with an intensity of 0 to 255 for an 8-bit black and white image or 0-255*3 for an RGB 8-bit color image). Lastly, another embodiment of the present invention is the use of the weighted value of the amplitude within the window of the artifact to understand how large or small they are in relation to other sorts of artifact. This can be accomplished straightforwardly by defining W(xᵢ)=Summation over j for all xⱼ*p(xⱼ) where xⱼ is the amplitude (for a time series) or intensity (for a 2D image) and p(xⱼ) is the probability or frequency of that value or intensity appearing within the artifact in each independent dimension(s).

Another embodiment of the present invention includes extracting the length L of the artifact in terms of individual samples from a time series, which can be calculated for each artifact by Lᵢ=xₗ−x_{f}. A follow-up embodiment considers the determination of the mean value of the signal over the artifact region, which is equal to the summation over j from the first to last sample of y(xⱼ) with the whole sum divided by N, where N=number of samples in the sum l-f. Another embodiment includes the nonlinearly calculated median value of the signal taken as the central value after an ascending or descending sort of the values has occurred. Another embodiment includes the standard deviation or square root of the variance (2^{nd} moment of the distribution) of the distribution of sample amplitudes from their previous experience. Likewise, the skewness (3^{rd} moment of the distribution) and the kurtosis (fourth moment of the distribution) are both straightforward embodiments of the present invention as variables to be extracted from the artifact samples and then utilized in the multi-modal data table as candidate predictors/features for use in univariate and multi-variate predictive modeling. After each of these is calculated on a per artifact basis, the number of artifacts N, the distribution of artifacts, the various moments of the distribution of various artifacts can thus be calculated and each of these can be extracted as another candidate diagnostic predictor or feature.

Of particular interest in the present invention is the evaluation of the relative position of artifacts in a data stream of interest relative to the presentation of external sensory and cognitive stimuli, or when a physical motion challenge is presented. For instance, if tones are supplied to the ears via ear buds, then noting the response of the brain bio-signals to the initiation and termination of the auditory tones to the auditory cortex is an embodiment of the present invention. Alternatively, another embodiment is the relative position of an artifact in one data stream when compared to other features of interest (good/correct answers or perhaps bad/incorrect answers, for instance if one is emotionally distressed) in other data streams that have been temporally synchronized in time.

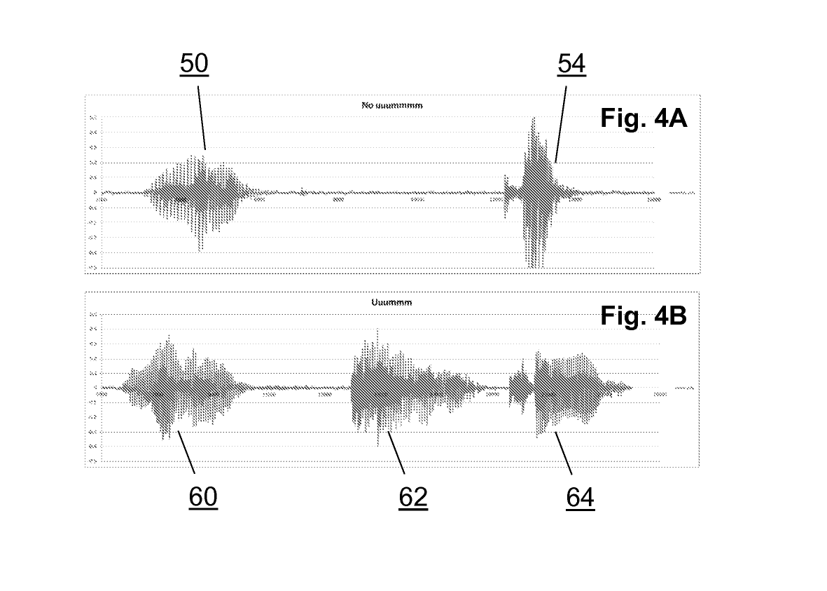

Of particular interest in accordance with the present invention is the evaluation of the auditory microphone data streams for heavy breathing or other audible signatures that reflect on the state of the brain of the subject. In FIG. 4A, one can see the raw microphone recording of amplitude as a function of time of a subject who counted the number “one” out loud 50, then paused, then counted “two” out loud 54, where the microphone or microphone array was acting as a biosensor data stream and was recorded. In FIG. 4B, one can see the raw microphone recording of a subject who counted the number “one” out loud 60, then paused and said “uummm” 62, then counted “two” out loud 64, where the microphone or microphone array was acting as a biosensor data stream and was recorded. In this instance, the presence of the “uumm” pattern could be noted and detected as an artifact and counted in an automated fashion to be extracted as a candidate biomarker for TBI and concussed individuals who are looking to buy some time when their brain is foggy in response to a question or cognitive or sensory challenge.

### EXAMPLES

While the above description contains many specifics, these specifics should not be construed as limitations on the scope of the invention, but merely as exemplifications of the disclosed embodiments. Those skilled in the art will envision many other possible variations that are within the scope of the invention. The following examples will be helpful to enable one skilled in the art to make, use, and practice the invention.

### Example 1

### Graded Symptom Checklist (Prophetic Example)

A non-limiting example is often illustrative. Consider a human subject who is being asked questions during a Graded Symptom Checklist (Cantu et al) style interview by a computer activated voice or the recording of a common voice (in order to standardize the presentation of questions). If a healthy normal subject is asked, they may just answer the question directly and provide an integer from zero to 6 (a correct response for this task for each pass through or question). On the other hand, imagine a concussed or traumatic brain injured subject who struggles to focus on each question being asked and utters an audible “uummm” just before each response because they unconsciously need a pause in time to reflect and generate a suitable answer. In this illustrative case, it would be very interesting and informative to analyze the microphone data stream for audible “uummm” artifacts, identify their location within the microphone data stream (time tᵢ for the iₜₕ “uummm”) and then calculate the relative timing of each artifact tᵢ to the time of the question t_{q} that would be asked of them. For example, if there is a temporal pattern which emerges such that concussed subjects typically say “uummm” as a mechanism to pause before answering questions and non-concussed subjects do not, then the number N of “uummms” or the frequency F of “uummms” or probability P of an “uummms” before any given response could become an extracted feature used to help predict to which class an unknown subject should be assigned or classified. In this way, the artifact becomes a putative predictor/feature with possible diagnostic information carried inside. Even simply counting the number N of “uummms” uttered could be indicative of a condition. Or alternatively, counting the number of “uummms” that occur more than some latency in time from a stimulus, in this case say 100 milliseconds after a question is asked, the number N₁₀₀ could be a more specific extracted feature with increased signal to noise ratio to predict in which class an unknown subject should be classified. Of course, once the artifact extracted features have been identified, then they can be looked at and investigated within traditional predictive analytical models that are univariate in nature as well as in multi-variate predictive models of the Support Vector Machine, Random Forest, Neural Nets, Discriminant Analysis variety, all well known in the art, in books such as Hastie, Tibshirani, Friedman, The Elements of Statistical Learning: Data Mining, Inference, and Prediction; 2^{nd} Ed, Springer (2009) or Duda, Hart, Stork, Pattern Classification; 2^{nd} Ed, Wiley Interscience, 2001.

### Example 2

### Increased Signal to Noise with the Balanced Error Scoring System (Prophetic Example)

Similarly, as a second non-limiting example, if one is asked to conduct the various static postures and positions of the Balanced Error Scoring System (BESS), the present state of the art is to have a certified athletic trainer supervise the human subject and click a stop-watch when the athlete begins and ends a posture, noting subjectively how many errors occurred according to the author's instructions. In an embodiment of the invention, the system marked the beginning and end of each 20 second posture based upon mouse clicks at a prescribed location (over a particular button) on the screen or from keyboard key strokes. In an exemplary embodiment, the microphone recorded data stream would automatically recognize the key word “Begin Now” and initiate an internal timer for 20 seconds, marking the end of the stance period with another time series marker as well as an audible “beep beep beep” to inform the human subject and test administrator that the time of that stance posture is now over. This form of automated data collection, across multiple modalities will enable more accurate and precise identification of significant bio signal extracted features. In fact, this aspect of the present invention is an important means of improving the signal to noise ratio of the data collected by eliminating those periods in time of data stream that are uncontrolled, not within task, and which essentially represent noisy data relative to the periods of time of interest when the subject is engaged in performing under the challenge or simulation of a given prescribed and physiologically focused task.

Either of these two modalities (mouse clicks on buttons at positions {x,y} on the screen at time t (x, y, t)/keyboard strokes on character keys at time t (char, t) or automated microphone based voice pattern recognition of “begin now” style commands) can be used to inclusively or exclusively gate the regions of data stream for analysis from one channel of information to enhance the signal in another channel of information. In this embodiment of the present invention, the assessment of static balance or dynamic balance by a human subject utilizes objective 3-axis accelerometer measurements (perhaps even 9 axis accelerometer, gyrometer, digital compass measurements), rather than the subjective opinion of a certified athletic trainer's judgment, to determine a level of balance or stability. Moreover, an important embodiment includes the analysis of the mean (1^{st} moment) of each axis of the accelerometer during the task period, the standard deviation (2^{nd} moment) of each axis during the task period, the skewness (3^{rd} moment) and even the kurtosis (4^{th} moment) of each of the 3 independent axis. A three dimensional surface can be constructed and the time averaged center of mass, center of gravity or centroid can be determined.

### Example 3

### Analysis of Eyes Open and Eyes Closed Blocks of Data for Artifacts (Actual Reduction to Practice)

Self-recordings were made in a 2 minute Eyes Closed, 2 minute Eyes Open, 2 minute Eyes Closed, 2 min Eyes Open fashion with a MindSetPro headset from NeuroSky. The 10 bit, 128 sample/sec data was loaded into MATLAB and software written to count the number N of artifacts in a block of data as well as count the number of artifact samples N_sam compared to the total block of data N_tot. The actual results can be seen in Table 1, which corresponds to the traces in FIG. 2. It is well established in the limited data before us that the Eyes Open conditions significantly increase the number and frequency of artifacts.

### Example 4

### Analysis of the Microphone Recording of a Subject Who Performed the King-Devick Neuro-Ophthalmologic Saccade Card Task

As part of the concussion battery at several company clinical sites, subjects are asked to read numbers off cards as fast as they can moving from left to right, top to bottom, without errors. It was observed that some concussed athletes appear to buy time by pausing and audiblizing “uummm” as if to buy time when cognition is not working properly. We then set out to characterize what an “umm” looks like in the microphone channel which can be seen in FIG. 4B at 62. The upper trace FIG. 4A does not contain an “uumm” as the gap between 50 and 54 is clear. Thus, in this instance, counting “uumms” could create an artifactual voice based extracted feature which is either a standalone or component of a multi-variate predictive statistical model.

Those skilled in the art will appreciate that the invention may be applied to other applications and may be modified without departing from the scope of the invention. Accordingly, the scope of the invention is not intended to be limited to the exemplary embodiments described above, but only by the appended claims.

What is claimed:

1. An analysis method for pre-processing biological sensor data from one of a plurality of concurrently collected independent biological sensor data streams, comprising:
identifying and flagging areas of artifact in said one biological sensor data stream; and
analytically characterizing features of the artifact as candidate predictor variables for predictive statistical models for analysis of said plurality of biological sensor data streams.

2. An analysis method as in claim 1, wherein said areas of artifact are used as time markers for analysis of said biological sensor data streams.

3. An analysis method as in claim 1, wherein a flagged artifact in said one biological sensor data stream is used to temporally gate data from another biological sensor data stream.

4. An analysis method as in claim 1, wherein the flagged artifact results from a manual action of a patient during a neuropsychiatric, neuropsychological, or cognition test.

5. An analysis method as in claim 4, wherein the manual action of the patient comprises clicks on one or more buttons or key strokes on one or more keys of a keyboard to mark times at the beginning and end of a time frame or period of interest.

6. An analysis method as in claim 1, wherein the artifact is automatically flagged without patient input.

7. An analysis method as in claim 6, wherein an acoustic microphone time series is analyzed to automatically determine when the first value is read and when a last value is read during oral testing of a patient.

8. An analysis method as in claim 1, wherein analytically characterizing features of the artifact comprises analyzing the artifact data for putative predictor variables to create additional features to be used as putative diagnostic information alone or to be used in development of multi-variate predictive statistical models.

9. An analysis method as in claim 1, wherein identifying and flagging areas of artifact comprises extracting a number N of artifacts in a block of data.

10. An analysis method as in claim 1, wherein identifying and flagging areas of artifact comprises determining a set of locations of artifacts within one-dimensional or two-dimensional data stream and recording the set of locations to a storage device.

11. An analysis method as in claim 1, wherein identifying and flagging areas of artifact comprises determining a central value of an artifact for a one-dimensional data stream or an equivalent for each dimension in a two-dimensional data stream.

12. An analysis method as in claim 1, wherein identifying and flagging areas of artifact comprises using a weighted value of an amplitude within a window of an artifact to understand how large or small the artifact is in relation to other artifacts.

13. An analysis method as in claim 1, wherein identifying and flagging areas of artifact comprises calculating distribution of extracted lengths L of the artifact in terms of individual samples from a time series, where the distribution is calculated for each artifact i by Lᵢ=xₗ−X_{f}.

14. An analysis method as in claim 13, wherein identifying and flagging areas of artifact comprises determining a mean value of the data in the one sensor data stream signal over a region of the artifact.

15. An analysis method as in claim 13, wherein identifying and flagging areas of artifact comprises using a nonlinearly calculated median value of values in the one sensor data stream within an artifact window taken as a central value after an ascending or descending sort of the values in the one sensor data stream has occurred.

16. An analysis method as in claim 1, wherein identifying and flagging areas of artifact comprises calculating a standard deviation and higher order moments of a distribution of sample amplitudes from zones in the one sensor data stream containing an artifact.

17. An analysis method as in claim 16, further comprising calculating a standard deviation and higher order moments of a distribution of sample amplitudes on a per artifact basis, a number of artifacts N, a distribution of artifacts, and various moments of the distribution of various artifacts for possible extraction as a candidate predictor variable.

18. An analysis method as in claim 1, further comprising using a relative position of artifacts in a first sensor data stream relative to presentation of external sensory and cognitive stimuli presentation of a physical motion challenge to a patient to identify a feature of interest in a second sensor data streams that has been temporally synchronized with the first sensor data stream.
